(19) United States
(12) Patent Application Publication
(10) Pub. No.: US 2023/0165494 A1
(43) Pub. Date: Jun. 1, 2023

(54) PROCESSING OF DRIED BLOOD SAMPLES ON LATERAL FLOW PAPER

(71) Applicant: Labonovum B.V., Limmen (NL)

(72) Inventor: Dennis Cornelis Wilhelmus POLAND, Limmen (NL)

(21) Appl. No.: 17/922,760

(22) PCT Filed: May 10, 2021
(86) PCT No.: PCT/NL2021/050301

§ 371 Date: Nov. 1, 2022

(30) Foreign Application Priority Data
May 8, 2020 (NL) 2025534
May 8, 2020 (NL) 2025535

(51) Int. Cl. A61B 5/15

(52) U.S. Cl. CPC A61B 5/150358; A61B 5/150343; A61B 5/150824; A61B 5/150755

(57) ABSTRACT

A grinder (220) is provided for use in a method of processing a piece of lateral flow paper, comprising a grinder body (222) defining a grinder storage volume (226) with a top opening for accessing the grinder storage volume and one or more through holes (228) providing a fluid connection through the grinder body into the grinder storage volume, wherein the grinder body further comprises one or more grinding protrusions (229) at an outer surface of the grinder body. Furthermore, a sample container (200) is provided for storing a blood sample provided on a piece of lateral flow paper, comprising a sample container body with a base part, a cylindrical wall extending from the base part and defining a storage volume with an opening for accessing the storage volume, wherein the base part comprises one or more protrusions (209) extending into the storage volume.

### TECHNICAL

The aspects and embodiments thereof relate to the field of collecting and processing of blood samples.

### BACKGROUND

NL2020386 discloses a device for collecting a sample of blood, comprising an elongated frame, arranged to hold a strip of lateral flow paper. The frame comprises a receiving opening arranged to expose a receiving section of the strip of lateral flow paper such that the sample of blood can be provided directly to the receiving section through the receiving opening.

NL2020386 further discloses a method for separating blood serum from a sample of blood comprising providing the sample of blood to a receiving section a strip of lateral flow paper, wherein the lateral flow paper is arranged to transport the sample of blood from the receiving section through a separation section, wherein in the separation section blood cells are separated from the blood serum.

### SUMMARY

In the known device for collecting a sample of blood, the frame can be opened by hinging two frame parts away from each other. When the frame is opened, the piece of lateral flow paper can be removed.

It is preferred to provide a device for collecting a sample of blood which allows for more automated processing of the sample of blood. Such more automated processing may for example be faster and/or more economical.

A first aspect provides a device for collecting a sample of blood comprising a paper holder arranged to hold a piece of lateral flow paper in a paper holder storage volume, and a paper cover body for covering at least part of the piece of lateral flow paper in a paper cover body storage volume, wherein the paper holder is releasably connected to the paper sample cover body such that in a connected state, the paper holder storage volume is provided adjacent to the paper cover body storage volume and in a disconnected state, the paper holder can be moved away from the paper cover body.

Releasably connected may imply that the paper holder may be disconnected from the paper sample cover body by a human and/or machine. For example, one more tools may be required for releasing the paper holder from the paper sample cover body. In particular embodiments, for releasing the paper holder from the paper sample cover body, the releasing is non-destructive.

Lateral flow paper is paper comprising fibres oriented in a particular direction, parallel to one another. Such paper may comprise cellulose fibres constituting the paper and oriented glass fibres which glass fibres are provided parallel to one another. Such paper is for example used to separate blood cells from blood serum of a sample of blood.

When the sample of blood is provided to a piece of lateral flow paper, blood serum provided in the sample of blood is transported through the lateral flow paper. During this transport, the blood serum is separated from the red and/or white blood cells by virtue of the properties of the lateral flow paper. The serum flows further than the red and/or white blood cells, which remain in a section defined as a separation section of the strip of lateral flow paper.

Lateral flow paper may be fragile and it's effectiveness in separating blood serum from a sample of blood may be at least partially comprised when handled incorrectly. Incorrect handling may be applying to much pressure to the lateral flow paper, touching the paper with dirty or greasy fingers, accidentally tearing the paper, or any other form of handling which may comprise the working of the lateral flow paper.

The device according to the first aspect comprises a paper cover body for protecting the piece of lateral flow paper held by the paper holder. Hence, the paper cover body may be arranged to fully or at least partially enclose the lateral flow paper when the paper holder holding the piece of lateral flow paper is in use connected to the paper cover body. Preferably, the piece of lateral flow paper is only in direct contact with the paper holder and not with other parts of the device. Optionally, support points may be available to prevent the piece of lateral flow paper from fluttering.

The device may comprise a sample container holder which is connected or at least connectable to the paper sample cover body and arranged to hold a sample container. As such, a particular sample container may be associated with the device. The sample container remains associated with the device and with the blood sample collected by device at least as long as the sample container remains connected with the sample container holder.

The device may further comprise a cartridge with a cartridge body arranged for housing at least part of the paper cover body, and wherein the paper holder and paper cover body in connected state can be moved at least partially into and out of the cartridge body between a transport state wherein at least part of the paper holder is cover by the cartridge body, and an open state wherein the at least part of the paper holder is exposed. The cartridge may provide additional protection to the piece of lateral flow paper.

The sample container may be embodied as a test tube comprising a top opening. When held in the sample container holder, the top opening may face towards the paper holder.

The cartridge body may comprise a window, the test tube may comprise a marking on an outside surface, and in the transport state, the window may be aligned with at least part of the marking. The marking may comprise a barcode, QR-code, one or more letters and or digits, or any combination thereof. With the window aligned with at least part of the marking in the transport state, the marking may be visible for a user and/or a sensor arranged to detect the marking such as a barcode scanner and/or computer vision system.

The device may be provided with or without the piece of lateral strip. Hence, the device may comprise a paper strip as a piece of lateral flow paper, which paper strip is at a proximal end held by the paper holder, and of which a distal end extends into the test tube in the connected state of the paper holder and the paper sample cover body.

A second aspect provides a method of processing a sample of dried blood on a piece of lateral flow paper, comprising disconnecting a paper holder from a paper cover body to expose part of a piece of lateral flow paper held by the paper holder, on the exposed part of the lateral flow paper, determining a transition area between a first area with red blood cells and a second area substantially without red blood cells, and cutting the piece of lateral flow paper based on the detected transition area to obtain a part of the lateral flow paper comprising separated blood serum.

The method according to the second aspect may be particularly applied to process a sample of dried blood collected by a device according to the first aspect.

Determining of the transition area may be performed by an optical sensor unit arranged for sending an optical signal to the lateral flow paper and receiving a reflected optical signal from the lateral flow paper. Hence, the different colours of different components of the dried blood samples may be used to automatically cut the piece of lateral flow paper at the desired position.

The method may further comprise depositing the cut off piece of lateral flow paper in a sample container, and adding a buffer fluid to the lateral flow paper in the sample container. For example, the depositing may be constituted by virtue of gravity pulling down on cut off piece of lateral flow paper.

The method may further comprise disconnecting the paper holder from a sample container holder holding the sample container. In particular when the cut off piece of the lateral flow paper is deposited into the sample container, the paper holder may be discarded. As an option, the left-over piece of the lateral flow paper still held in the paper holder may be used for a different method of processing a sample of blood of a piece of lateral flow paper.

After, before or while the cut off piece of the lateral flow paper is deposited into the sample container, a buffer liquid may be added into the sample container. In an optional further method step, a grinder may be placed into the sample container after the cut-off piece of lateral flow paper and the buffer fluid are deposited into the sample container, and using the grinder for mixing the cut-off piece of lateral flow paper and the buffer fluid.

After having used the grind for mixing the cut-off piece of lateral flow paper and the buffer fluid, at least part of the buffer fluid may be retrieved from the grinder. The fluid may be retrieved from the grinder because the grinder comprises one or more through holes providing a fluid connection through the grinder body into the grinder storage volume.

A third aspect provides a grinder for use in a method of processing a piece of lateral flow paper, for example for use in a method according to the second aspect. The grinder comprises a grinder body defining a grinder storage volume with a top opening for accessing the grinder storage volume, one or more through holes providing a fluid connection through the grinder body into the grinder storage volume, wherein the grinder body further comprises one or more grinding protrusions at an outer surface of the grinder body, which one or more grinding protrusions face away from the grinder storage volume.

The grinder body may comprise a base part and a cylindrical wall protruding from the base part, and at least one of the one or more through holes may be provided through the base part.

As an option, the one or more grinding protrusions may be provided adjacent to the one or more through holes.

A fourth aspect provides a sample container for storing a blood sample on a piece of lateral flow paper, which may be shaped such that it may be held in a sample container holder of a device according to the first aspect. The sample container may comprise a sample container body with a base part, a cylindrical wall extending from the base part defining a storage volume with an opening for accessing the storage volume, wherein the base part comprises one or more protrusions extending into the storage volume.

The one or more protrusions may be at least partially spherical. The spherical shape of the one more protrusions may form a contact area for a grinder with a low surface area, which may reduce the force required to rotate the grinder inside storage volume.

The sample container may further comprise a foot part extending from the base part away from the opening. The foot part may define a hollow chamber with a chamber opening on an opposite side of the sample container than the opening of the storage volume.

A fifth aspect provides a kit of parts to form a sample container assembly, comprising a grinder according to the third aspect, and a sample container according to the fourth aspect, wherein the sample container is arranged to receive at least part of the grinder body in the storage volume.

### BRIEF DESCRIPTION OF THE FIGURES

In the figures,

FIGS. 1A and 1B show an embodiment of a device for collecting a sample of blood;

FIGS. 1C and 1D show a further embodiment of the device for collecting a sample of blood;

FIG. 2A shows an exploded view of an embodiment of a device for collecting a blood sample;

FIG. 2B shows a cross-sectional view of an embodiment of a device for collecting a blood sample;

FIG. 3A shows a sample container assembly;

FIG. 3B shows a cross-section view of a grinder;

FIG. 3C shows an exploded view of a sample container assembly; and

FIG. 4 shows a detailed cross-sectional view of part of a test tube as an example of a sample container.

### DETAILED DESCRIPTION OF THE FIGURES

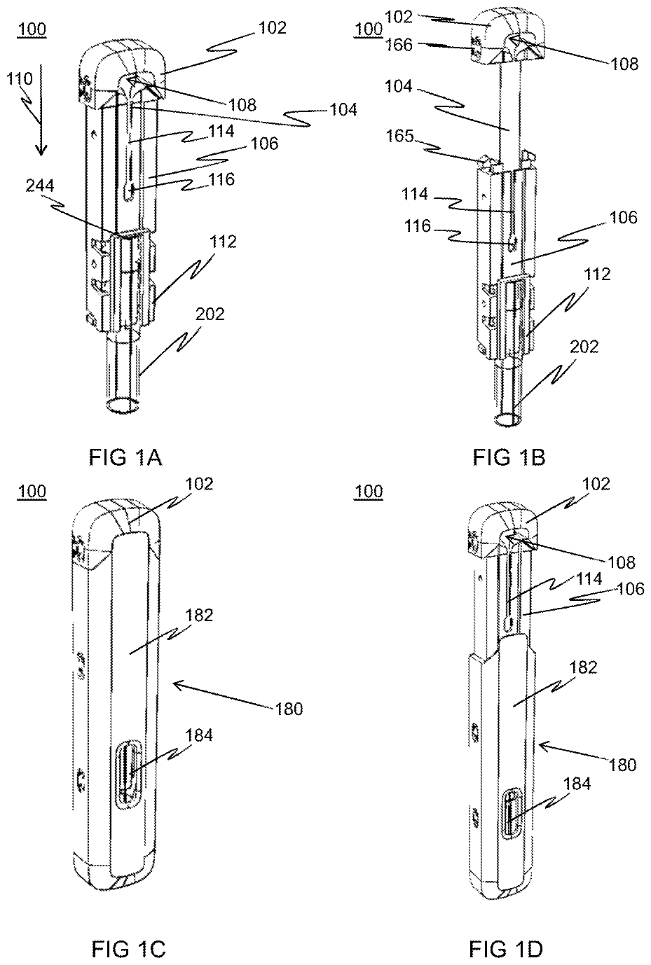

FIG. 1A shows an embodiment of a device 100 for collecting a sample of blood, wherein the device 100 is shown in a connected state. The device 100 comprises a paper holder 102 arranged to hold a paper strip 104 as a piece of lateral flow paper. The device 100 further comprises a paper cover body 106 for covering at least part of the paper strip 104. FIG. 1B shows the device 100 of FIG. 1A in a disconnected state, in which the paper strip 104 can be at least partially removed from the paper cover body 106.

In particular, in the disconnected state as shown in FIG. 1B, an additional portion of the paper strip 104 is exposed compared to the connected state as shown in FIG. 1A. In particular, this additional portion was in the connected state of the device 100 covered by the paper cover body 106. Hence, the additional portion may be identified as the portion of the paper strip 104 not exposed in the connected state, but exposed in the disconnected state.

The paper holder 102 comprises a reception opening 108 providing a flow path to a proximal end of the paper strip 104. When a sample of liquid blood is provided to the proximal end of the paper strip 104, part of the blood sample may migrate through the paper strip 104 towards a distal end of the paper strip 104, in a direction indicated with arrow 110. The reception opening 108 may be provided by a through hole, cavity, and/or any other type of opening in the paper holder 102.

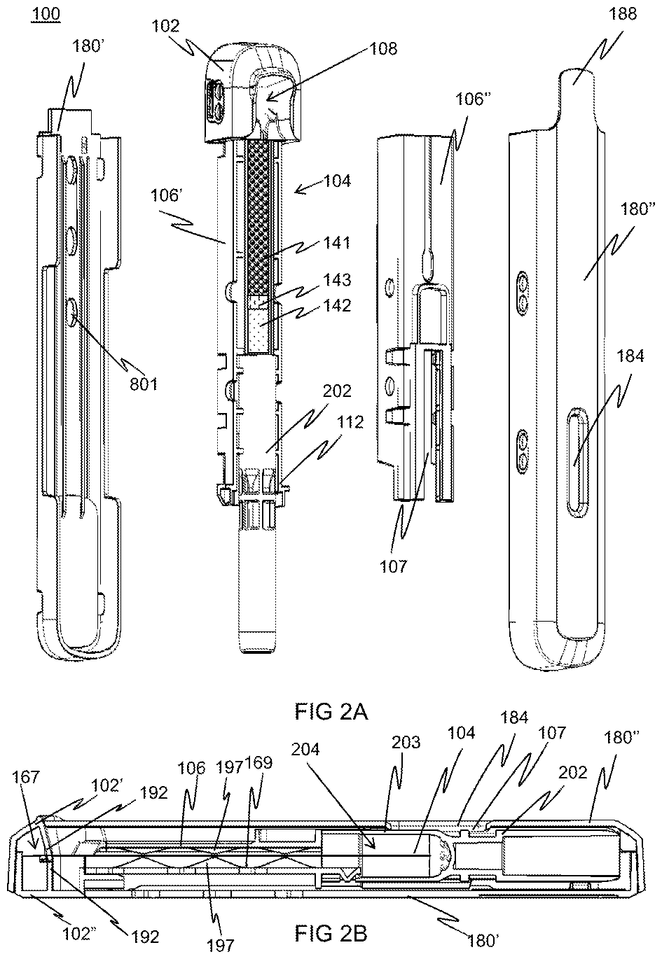

The reception opening 108 ends in a paper holder storage volume 167 of the paper holder 102. This paper holder storage volume 167 may be defined as a volume defined by the paper holder 102 in which lateral flow paper may be contained. The paper holder storage volume 167 is in FIG. 2B shown adjacent to a paper cover body storage volume 169, as the device 100 is in FIG. 2B is shown in a connected state. In the connected state, with the paper holder storage volume 167 adjacent to the paper cover body storage volume 169, the paper strip 104 may be accommodated in both the paper holder storage volume 167 and the paper cover body storage volume 169 in a contiguous way.

The paper holder 102 and the paper cover body 106 are preferably releasably connected in the sense that they may be separated without being damaged. The connection may be provided by means of hinges, snap-fit connections, press-fit connections, other or a combination thereof.

In a released or disconnected state, the paper holder 102 and the paper cover body 106 are moved away from another, separating the paper holder storage volume 167 from the paper cover body storage volume 169. With the paper strip 104 being fit in the paper holder storage volume 167 of the paper holder 102, a part of the paper strip 104 is exposed, which exposed part was provided in the paper cover body storage volume 169 in the connected state, as described above.

As an option, the device 100 further comprises a sample container holder 112 arranged to hold a test tube 202 as an example of a sample container. The test tube 202 comprises an opening 244 for receiving at least part of the paper strip 104 into a storage volume of the test tube 202. For example, the test tube 202 may be clamped by the sample container holder 112.

Preferably, the test tube 202 is releasably connected to the sample container holder 112 such that the test tube 202 may be disconnected without damaging the test tube 202. Damage to the test tube 202 may possibly result in an undesired contamination of an inside of the test tube 202 and/or content inside the test tube 202 may leak out.

In particular, the sample container holder 112 is arranged to hold the test tube 202 in an orientation wherein the opening 244 of the test tube 202 faces towards the paper holder 102, and thus, in use, towards the paper strip 104. As such, when the device 100 is in use oriented substantially vertical—i.e. substantially parallel to the gravity vector—with the opening 244 facing upwards, part of the paper strip 104 may drop into the test tube 202. This part of the paper strip 104 may be either released from the paper holder 102 or from another part of the paper strip 104 which may remain attached to the paper holder 102.

In the embodiment of FIGS. 1A and 1B, the paper cover body 106 and the sample container holder 112 are provided by a single component. In other embodiments, the paper cover body 106 and the sample container holder 112 may be separate components which are further optionally connectable to each other.

The paper cover body 106 as shown in FIGS. 1A and 1B comprises an optional slit 114 as a viewing window for viewing part of the paper strip 104. The slit 114 may extend between the reception opening 108 and a further optional indication opening 116 which is broader than the width of the slit 114. Through the slit 114 and the optional indication opening 116, a visual indicator may be provided to a user of the device 100 of the amount of blood provided to the paper strip 104.

Since the slit 114 is optional, it will be appreciated that embodiments of the device 100 are envisioned with the slit 114. As an alternative to the slit 114, one or more openings may be provided to form the viewing window for viewing part of the paper strip 104. The one or more openings may be formed as through holes, may be formed by an at least partially transparent part, or as a combination thereof.

The paper cover body 106 may be connectable to the paper holder 102. For example, as shown in FIG. 1B, the paper cover body 106 as a first of the paper cover body and the paper holder comprises one or more latches 165 arranged to latch behind one or more openings 166 comprised by the paper holder 102 as a second of the paper cover body and the paper holder. The one or more latches 165 may be hingedly connected to the paper cover body 106.

Through the one or more openings 166, the one or more latches 165 may be accessible when the paper holder 102 is connected to the paper cover body 106. As such, a machine may be used to engage the one or more latches 165 through the one or more openings 166 to disconnect the paper holder 102 from the paper cover body 106.

FIGS. 1C and 1D show a further embodiment of the device 100 for collecting a sample of blood, which embodiment comprises an optional cartridge 180. The cartridge 180 comprises a cartridge body 182 arranged to receive at least part of the paper cover body 106 and optionally also also arranged to receive at least part of the paper holder 102.

In FIG. 1C, the device 100 is in a transport state wherein preferably non of the paper strip 104 is exposed. Hence, in the transport state, the paper strip 104 may be protected from outside influences which may otherwise when the paper strip 104 would be exposed for example damage the paper strip 104 or contaminated the blood sample.

In FIG. 1D, the device 100 is shown in an open state. To transition between the transport state of FIG. 1C and the open state of FIG. 1D, for example, the paper holder 102 and the paper cover 106 may be at least partially slit out of the cartridge body 182. The cartridge body 182 may be an elongated body, and the sliding direction may be substantially parallel to an elongation direction of the cartridge body 182. The cartridge body 182 and the paper cover 106 and/or the paper holder 102 may in the open state and/or the closed state be releasably fixated relative to one another to restrict movement of the various parts relative to one another. The fixating may be provided by notches and indentations, respectively, where notches may snap in and out of the indentations to restrict and enable movement, respectively.

In the open state, the device 100 may be arranged to receive the blood sample on the paper strip 104 through the opening 108. After the blood sample is received, the paper holder 102 may be moved towards the cartridge 180 to put the device 100 in the transport state shown in FIG. 1C.

As an option, the cartridge body 182 may comprise a window 184, which when present provides a passage for light through the cartridge body 182. As such, a part of the sample container 202 held in the sample container 112 may be aligned with the window 184 when the device 100 is in the transport state and/or in the open state. This part of the sample container 202 may comprise a marker such as a barcode, QR code, text, any other type of marking, or any combination thereof.

When the sample container 202 comprises such a marker, which is located to align with the window 184, even in the transport state of the device 100 the sample container 202 may be identified.

Also in embodiments of the device 100 without the cartridge 180 or the window 184, having a marker on the sample container 202 may be advantageous. The marker may allow automatic identification of the sample container 202, for example by a computer device with a scanner arranged to scan the marker.

In particular with embodiments of the device 100 comprising a sample container 202 held in the sample container holder 106, which in turn may be connected to the paper holder 102, the same sample container 202 remains associated with the same piece of lateral flow paper held in the paper holder 102. It may be more convenient to provide a sample container with a marker than a piece of lateral flow paper, which in general is more prone to being damaged.

FIG. 2A shows an exploded view of an embodiment of the device 100 for collecting a blood sample. The device 100 comprises the paper holder 102 holding the paper strip 104 which is in this figure shown after having been provided with a blood sample.

The paper holder 106 in this embodiment comprises two shell parts: a bottom paper holder part 106′ and a top paper holder part 106″ which are connectable to each other. As an option, the top paper holder part 106″ comprises an opening 107 arranged to allow light from passing between the window 184 and the part of the sample container 202 aligned with the window 184 in a particular state of the device 100. As such, the top paper holder part 106″ may not block viewing of the part of the sample container 202 which is aligned with the window 184.

The device 100 shown in FIG. 2A further comprises the optional cartridge 180, which in this embodiment comprising two shell parts: a bottom cartridge part 180′ and a top cartridge part 180″. The two shell parts may be connected to each other to form the cartridge body 182. As an option, the top cartridge part 180″ comprises the window 184. As an even further option, the top cartridge part 180″ comprises a reception opening cover 188 arranged to substantially cover the reception opening 108 in the transport state of the device 100.

The optional cartridge 180 may comprise one or more ventilation holes 801, for example in the bottom cartridge part 180′. By virtue of the one or more ventilation holes 801, the blood sample on the paper strip 104 may dry quicker.

When a blood sample is provided to the paper strip 104 through the reception opening 108, after an amount of time, a first area 141 and a second area 142 may be present on the paper strip 104. The first area 141 comprises red blood cells which are filtered from the sample of blood by the lateral flow paper. After some flow distance, substantially all red blood cells are filtered out and hence the second area 142 may comprises substantially no red blood cells. The second area 142 may extend towards the distal end of the paper strip 104—or not.

The second area 142 may comprise serum, including compounds usually present in serum, including, but not limited to proteins and/or amino acids, minerals, fats, other or a combination thereof. A transition area 143 is present between the first area 141 and the second area 142. The transition area 143 may be a particularly small area resembling a line perpendicular to a flow direction of the blood sample through the paper strip 104. The transition area 142 generally comprises a combination of blood cells and serum.

As visible in FIG. 2A, the second area 142 may extend into the sample container 202, whereas as an option the first area 141 and the transition area 143 do not extend into the sample container 202. In another embodiment, the second area 142 does not extend into the sample container 202 and in yet another embodiment, the first area 141 extends into the sample container.

The person skilled in the art will appreciate that the size of the first area 141, second area 142 and transition area 143 may depend on properties of the lateral flow paper such as width and thickness, and on the volume of the blood sample provided to the lateral flow paper as well as where on the paper 104 strip the sample is provided.

FIG. 2B shows a cross-sectional view of an embodiment of the device 100 for collecting a blood sample. In particular visible in FIG. 2B is that a part of the paper strip 104 is inserted into a storage area 204 of the sample container 202. The paper strip 104 is held in place by the paper holder 102.

Also shown in FIG. 2B is the option that the paper holder 102 comprises two shell parts: a top paper holder part 102′ and a bottom paper holder part 102″. The paper strip 104 may be clamped between two clamp halves 192. As an option, the paper strip 104 may be perforated at one or more locations by at least one of the clamp halves 192 to further connect and fix the paper strip 104 to the paper holder 102.

As an option, the paper holder 102 is at least partially shaped as a scraper, against which a user may scrape his finger. When this finger is punctured to allow blood to flow out of the finger, using the scraper, blood may be guided into the reception opening 108 onto the paper strip 104.

As an even further option, at least one of the top paper holder part 102′ and the bottom paper holder part 102″ comprises one or more support protrusions 197. Preferably, at a distal end of a support protrusion 197 which contacts the paper strip 104, the contact area is as small as possible to prevent blocking the flow of blood through the paper strip 104. Hence, the support protrusions may be for example shaped at least partially as a pyramid, triangle, sphere, any other shape, or any combination thereof. Different support protrusions 197 may be shaped differently.

When both the top paper holder part 102′ and the bottom paper holder part 102″ comprise a support protrusion 197, these support protrusions 197 may be aligned such that they contact the paper strip 104 at opposite sides substantially at the same location. As such, the paper strip 104 may be clamped at this location, as is visible in FIG. 2B.

In general, two or more components may be connected by virtue of a clamping connection, forced connection with for example one or more springs, a shape connection wherein for example a part of a first component latches on or fits into to a second component, a glued connection, a welded connection, any other means of connection, or any combination thereof. This in particularly applies to two or more shell parts which may form components such as the paper holder, paper cover body and cartridge.

The two shell parts which may form at least part of the paper cover body 106, as shown in FIG. 2B, may define a paper storage volume 169 in which at least part of the paper strip 104 may be placed.

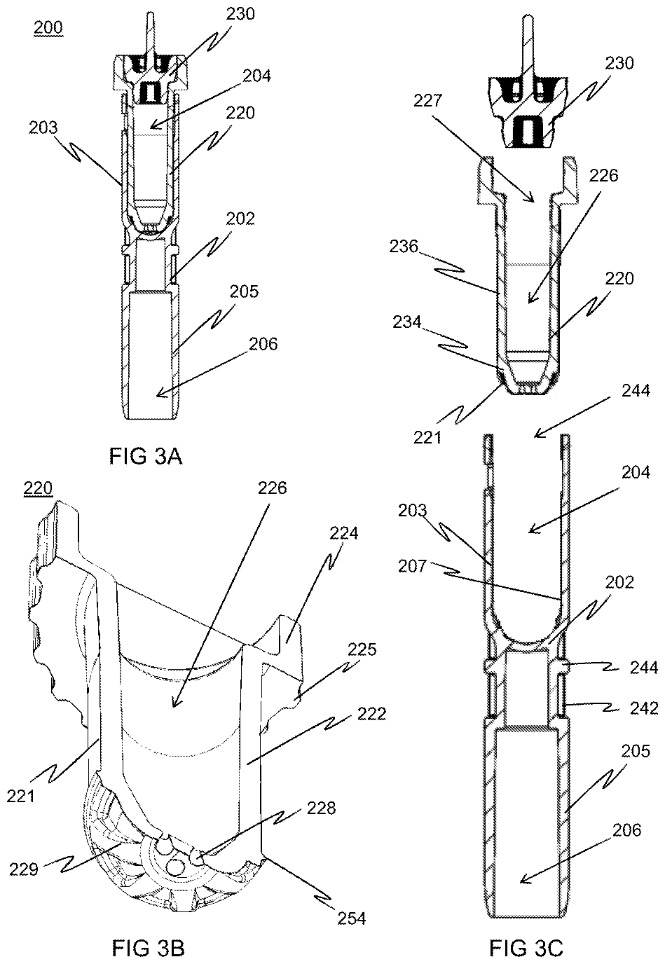

FIG. 3A shows a sample container assembly 200, comprising a test tube 202 as an example of a sample container. FIG. 3B shows a cross-section view of a grinder 220, and FIG. 3C shows an exploded view of the sample container assembly 200 of FIG. 3A.

The test tube 202 comprises a storage volume 204 provided in a storage part 203 of the test tube 202. As an option, the test tube 202 further comprises a foot part 205 providing a hollow chamber 206. The test tube 202 may be placed on a pin by sliding the foot part 205 over such a pin such that the pin is inserted into the hollow chamber 206. The opening of this hollow chamber 206 faces away from the storage volume 204. Test tubes 202 without the hollow chamber 206 are also envisioned. The foot part 205 may be used to shape the test tube 202 to comply with standard sizes which are compatible with conventional laboratory equipment.

The grinder 220 comprises a grinder body 222 and an optional gripper part 224 connected to the grinder body 222. One or more protrusions 225 of any shape may extend from the gripper part 224 to improve gripping of the grinder 220, for example with a persons fingers of which a gripper part comprised by a machine. The gripper part 224 may have a larger outer diameter than the gripper body 222, as visible for example in FIG. 3B.

The grinder body 222 defines a grinder storage volume 226 and comprises a top opening 227 at an—in use—top side of the grinder body 222. The grinder body 222 further comprises one or more through holes 228, which provide a fluid connection through the grinder body 222 into the grinder storage volume 226. Preferably, the one or more through holes 228 are provided in an in use bottom halve of the grinder body 222. Even more preferably, the one or more through holes 228 are provided substantially as far away as possible from the top opening 227.

The grinder body 222 for example comprises a base part 234 and a cylindrical wall 236 protruding from the base part 234. For example, the base part 234 may be at least partially flat, spherical, have any other shape, or any combination thereof.

As a further option, on an outer wall of the grinder body, one or more grinding protrusions 229 are provided extending away from the storage volume 226. In the embodiment of FIG. 3B, the grinding protrusions are provided adjacent to the through holes 228.

The sample container assembly 200, as a further option, comprises a cap 230 arranged for substantially liquid-tightly sealing off the top opening 227 of the grinder body 222. At least one of the cap 230 and the grinder body 222 may comprise a resilient material arranged to elastically deform to correspond to the shape of the other of the cap 230 and the grinder body 222, to form a liquid-tight seal.

In use, a piece of lateral flow paper comprising blood serum, as an example of a blood sample, may be placed into and/or onto the bottom of the storage volume 204 of the test tube 202. In order to fit the piece of lateral flow paper into the storage 204, a pestle may be used to push the piece of paper entirely into the storage volume 204. The pestle may be automatically operated by a machine. Furthermore, a liquid such as a buffer liquid may be provided to the storage volume 204 as well.

When the piece of lateral flow paper and the liquid have been placed inside the storage volume 204, the grinder 220 may be placed into the storage volume 204 as well. Hence, an outer shape of at least part of the grinder body 222 may correspond to the test tube 202 such that it may fit at least partially into the storage volume 204. Preferably, the outer shape of the grinder body 222 is substantially complementary to the storage volume 204.

When the grinder 220 is placed into the storage volume 204, the piece of lateral flow paper is positioned between an outer wall of the grinder body 222 and an inner wall 207 of the storage volume 204. By rotating the grinder 220 inside the storage volume 204, for example around an axis substantially perpendicular to the top opening 227, the piece of lateral flow paper is agitated within the liquid. This may enhance mixing of the piece of lateral flow paper and the liquid, in particular with blood serum components present on the lateral flow paper and the liquid. In examples, at least part of the piece of lateral flow paper may break into smaller pieces and/or at least partially disintegrated to enhance at least partial dissolution of at least part of serum components in the liquid added.

The method of grinding may further comprise may further comprise moving the grinder 220 at least partially in and out of the storage volume 204 during and/or between rotating the grinder 220, i.e. a rotational and/or lateral movement of the grinder 220 relative to the storage volume 204.

By virtue of the one or more through holes 228 of the grinder body 222, the liquid with dissolved components of the blood serum may enter the grinder storage volume 226. After some time, the fluid level inside the grinder body 222 may correspond to the fluid level inside the storage volume 204 of the test tube.

Finally, for example, the liquid inside the grinder body 222 may be extracted from the grinder body 222 using for example a pipette through the top opening 227. This liquid may be substantially free of lateral flow paper, which may be left behind between the outer wall of the grinder body 222 and the inner wall 207 of the storage volume 204.

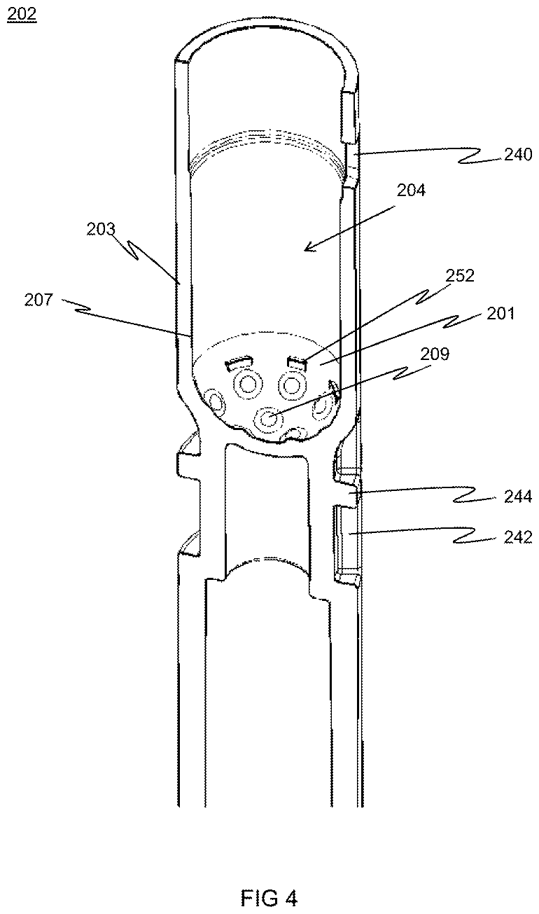

FIG. 4 shows a detailed cross-sectional view of part of the test tube 202 as an example of a sample container. As an option also applicable in other embodiments of sample containers, the inner wall 207 of the test tube 202 is provided with one or more protrusions 209 protruding into the storage volume 204. In the particular embodiment of FIG. 4, the protrusions 209 are provided in a curved bottom part 201 of the storage part 203 of the test tube 202.

The protrusions 209 may aid in the cutting, tearing, rupturing or otherwise disintegration of a piece of lateral flow paper placed between the protrusions 209 and the outer wall 221 of the grinder 220. When the grinder 220 comprises one or more grinding protrusions 229, when the grinder 220 is placed into the storage volume 204, these grinding protrusions 229 may contact the protrusions 209 of the test tube 202, or may at least be aligned with the protrusions 209 of the test tube 202.

The protrusions 209 may additionally or alternatively be used to decrease a contact surface area between the grinder body 222 and the inner wall 207 of the storage area 204, which may reduce the friction force between the grinder body 222 and the inner wall 207 when the grinder body 222 is rotated relative to the inner wall 207. For example, one more more of the protrusions 209 may comprises an outer surface which is partially spherical. Such a spherical shape may be used to resemble a point contact with the grinder body 222, which has a particularly low contact surface area. In other embodiment, the protrusions may have differently curved shapes, facetted shapes, for example pyramid-like shapes, other, or a combination thereof.

As an even further option shown in FIG. 4, the test tube 202 comprises one or more stopping ridges 252 protruding into the storage volume 204 as abutments. The stopping ridges 252 may define a maximum penetration of the grinder 220 into the storage volume.

For example, as shown in FIG. 3B, the grinder 220 may comprise one or more O-rings 254. The one or more O-rings 254 may substantially prevent liquid from rising between the inner wall 207 of the storage volume and the outer wall 221 of the grinder towards the opening 224. The stopping ridges 252 may alternatively or additionally allow a substantially coaxial alignment between the grinder body 222 and the storage part 203 of the test tube 202. As such, the stopping ridges 252 may extend to a smaller radius than the inner wall 207 of the storage volume 204.

The test tube 202 as shown in FIG. 4 may as option comprise one or more features arranged to help fixate a position of the test tube 202, for example during the grinding process. For example, an upper indentation 240 may be provided, with which a rotational position of the test tube 202 may be fixated to prevent rotation of the test tube 202 around its elongation axis.

As another example, one or more ribs 242 may be provided on an outer surface of the test tube 202. The one or more ribs 242 may extend substantially radially from the test tube 202 and extend substantially parallel to an elongation direction of the test tube 202.

As a further option, a further rib 244 may be provided on the outer surface of the test tube 202. The further rib 244 extends substantially radially from the outer surface and at least partially circumferentially surrounds the outer surface of the test tube 202.

In general, the outer surface of the test tube 202 may be provided with any number of protrusions and/or indentations which may be engaged by one or more clamping devices to fixate a position and/or orientation of the test tube 202.

Next to the device 100 and the sample container 200, a method of processing a sample of blood on a piece of lateral flow paper is envisioned. The paper strip 104 is an example of such a piece of lateral flow paper. Embodiments of the method will be elaborated on in conjunction with the figures.

In a first method step, the paper holder 102 is released or disconnected from the paper cover body 106 to expose a paper strip 104 as a piece of lateral flow paper held by the paper holder 102. Prior to this first method step, the device 100 may be in the connected state shown in FIG. 1A, and after this first method step, the device 100 may be in the disconnected state shown in FIG. 1B.

When the device 100 comprises a cartridge 180, the cartridge 180 may be removed prior to the first method step or after the first method step. Hence, prior to the first method step, the paper holder 102 together with the paper cover body 106 may be pulled out of the cartridge, as for example shown in FIG. 1D.

In a second method step, on the exposed part of the paper strip 104, a transition between the first area 141 and the second area 142 and the transition area 143 in particular may be identified. In general, it may be sufficient to identify two of the first area 141, second area 142 and the transition area 143 to also identify the third of the first area 141, second area 142 and the transition area 143.

The paper strip 104 may be cut in a third method step. It may be preferred to analyse only blood serum, and/or it may be preferred to have an as less as possible number of red and/or white blood cells in a sample that is to be processed. By cutting the paper strip 104 based on the determined transition area, the second area 142 may be separated from the first area 141. Preferably, the paper strip 104 is cut below the first area 141, either just below the first area 141, in the transition area 143, just below the transition area 143 or within the second area 142. By using this way of cutting, a piece of paper substantially without red and/or white blood cells may be obtained. The first area 141 of the paper strip 104 may be used for different purposes than the second area 142, for example for DNA analysis or Hb-chromatography.

Since red blood cells reflect different wavelengths of light than blood serum, an optical sensor unit may be used to determine a location where to cut the paper as described above.

The optical sensor unit may be arranged for sending an optical signal to at least part of the lateral flow paper and for receiving a reflected optical signal from the lateral flow paper.

For example, the optical signal reflected by the paper strip 104 and compound provided therein comprises light with wavelengths corresponding to red blood cells, which may be indicative of light reflected from the first area. The amount of light with wavelengths corresponding to red blood cells reflected may be lower or even significantly lower in the transition area and the second area 142 on the paper strip. This point may be used to electronically identify a transition between the second area 142 and the first area 141. The cut location may be defined in the transition area or other the transition point—or at a pre-determined distance below the transition area or other the transition point.

As an optional method step, the length and/or surface area of the second area 142 may be determined or measured, for example using the optical sensor unit. Using data indicative of the length and/or surface area of the second area 142, the amount of liquid to be added to the test tube may be determined to have a specific dilution of the serum after grinding. The amount of liquid added is preferably proportional to the length of the paper that has been cut off, optionally with a particular offset of the paper taken into account.

By virtue of gravity and the weight of the cut off piece of paper, the cut off piece may fall into the sample container 202 when the device 100 is held substantially parallel to gravity vector, with the opening of het sample container facing upwards towards the cut off piece of paper.

In a preferred embodiment, the paper holder 102 is disconnected or released from the a paper cover body 106 and more preferably, in a direction parallel to the length of the paper cover body 106. In this way, the paper strip 104 is at least partially exposed, as discussed above. This allows the transition between the first area 141 and the second area 142 to be identified or more conveniently identified.

Furthermore, this allows the paper strip 104 to be cut as described above. After the cut, the part of the paper strip 104 not held by the paper holder 102, the part comprising no or virtually no red blood cells, falls in the sample container 202. Next, as discussed above, the paper part cut off may be pushed in the sample container 202, using for example a pestle.

Before or after the cut off piece of paper is deposited into the sample container 202, a buffer liquid may be added into the sample container. Next, the grinder, for example shown in FIG. 3B may be placed into the sample container with the cut off piece of paper and the liquid.

1. A device for collecting a sample of blood, comprising:
a paper holder arranged to hold a piece of lateral flow paper in a paper holder storage volume; and
a paper cover body for covering at least part of the piece of lateral flow paper in a paper cover body storage volume,
wherein the paper holder is releasably connected to the paper sample cover body such that in a connected state, the paper holder storage volume is provided adjacent to the paper cover body storage volume and in a disconnected state, the paper holder can be moved away from the paper cover body.

2. The device according to claim 1, further comprising a sample container holder which is connected to the paper sample cover body and arranged to hold a sample container.

3. The device according to claim 1, further comprising a cartridge with a cartridge body arranged for housing at least part of the paper cover body, and wherein the paper holder and the paper cover body in connected state can be moved at least partially into and out of the cartridge body between a transport state wherein at least part of the paper holder is covered by the cartridge body, and an open state wherein the at least part of the paper holder is exposed.

4. The device according to claim 2, further comprising a test tube as a sample container, which test tube is held in the sample container holder, and wherein the test tube comprises a top opening facing towards the paper holder.

5. The device according to claim 4,
further comprising a cartridge with a cartridge body arranged for housing at least part of the paper cover body, and wherein the paper holder and the paper cover body in connected state can be moved at least partially into and out of the cartridge body between a transport state wherein at least part of the paper holder is covered by the cartridge body, and an open state wherein the at least part of the paper holder is exposed,
wherein the cartridge body comprises a window, the test tube comprises a marking on an outside surface, and in the transport state, the window is aligned with at least part of the marking.

6. The device according to claim 4, further comprising a paper strip as a piece of lateral flow paper, which paper strip is at a proximal end held by the paper holder, and of which a distal end extends into the test tube in the connected state of the paper holder and the paper sample cover body.

7. A method of processing a sample of blood on a piece of lateral flow paper, comprising:
disconnecting a paper holder from a paper cover body to expose part of a piece of lateral flow paper held by the paper holder;
on the exposed part of the lateral flow paper, determining a transition area between a first area with blood cells and a second area substantially without blood cells; and
cutting the piece of lateral flow paper based on the determined transition area to obtain a cut off piece of the lateral flow paper comprising separated blood serum.

8. The method according to claim 7, wherein the determining of the transition area is performed by an optical sensor unit arranged for receiving a reflected optical signal from the lateral flow paper.

9. The method according to claim 7, further comprising depositing the cut off piece of lateral flow paper in a sample container, and adding a buffer fluid to the lateral flow paper in the sample container.

10. The method according to claim 9, wherein the cut-off piece of lateral flow paper is deposited into the sample container by virtue of gravity.

11. The method according to claim 9, further comprising disconnecting the paper holder from a sample container holder holding the sample container.

12. The method according to claim 9, further comprising placing a grinder into the sample container after the cut-off piece of lateral flow paper and the buffer fluid are deposited into the sample container, and using the grinder for mixing the cut-off piece of lateral flow paper and the buffer fluid.

13. The method according to claim 12, further comprising retrieving at least part of the buffer fluid from the grinder after the step of using the grinder for mixing the cut-off piece of lateral flow paper and the buffer fluid.

14. A grinder for use in a method of processing a piece of lateral flow paper, comprising:
a grinder body defining a grinder storage volume with a top opening for accessing the grinder storage volume; and
one or more through holes providing a fluid connection through the grinder body into the grinder storage volume,
wherein the grinder body further comprises one or more grinding protrusions at an outer surface of the grinder body, which one or more grinding protrusions face away from the grinder storage volume.

15. The grinder according to claim 14, wherein the grinder body comprises a base part and a cylindrical wall protruding from the base part, and wherein the one or more through holes are provided through the base part.

16. The grinder according to claim 14, wherein the one or more grinding protrusions are provided adjacent to the one or more through holes.

17. A sample container for storing a blood sample provided on a piece of lateral flow paper, comprising a sample container body with a base part, a cylindrical wall extending from the base part and defining a storage volume with an opening for accessing the storage volume, wherein the base part comprises one or more protrusions extending into the storage volume.

18. The sample container according to claim 17, wherein the one or more protrusions are at least partially spherical.

19. The sample container according to claim 17, further comprising a foot part extending from the base part away from the opening, which foot part defines a hollow chamber with a chamber opening on an opposite side of the sample container than the opening of the storage volume.

20. A kit of parts to form a sample container assembly, comprising:
the grinder according to claim 14; and
a sample container for storing a blood sample provided on a piece of lateral flow paper, comprising a sample container body with a base part, a cylindrical wall extending from the base part and defining a storage volume with an opening for accessing the storage volume, wherein the base part comprises one or more protrusions extending into the storage volume,
wherein the sample container is arranged to receive at least part of the grinder body in the storage volume.
